(19) United States
(12) Patent Application Publication
(10) Pub. No.: US 2007/0072920 A1
(43) Pub. Date: Mar. 29, 2007

(54) Phenylethylamine analogs and their use for treating glaucoma

(75) Inventors: Mark R. Hellberg, Arlington, TX (US); Abdelmoula Namil, Arlington, TX (US); Zixia Feng, Arlington, TX (US); Jennifer Ward, Justin, TX (US)

Correspondence Address: Alcon Research, Ltd.;Patrick M. Ryan TB4-8, IP Legal Department, 6201 So. Freeway, Fort Worth, TX, 76134-2099, US

(73) Assignee: Alcon, Inc.

(21) Appl. No.: 11/525,975

(22) Filed: Sep. 22, 2006

Related U.S. Application Data
(60) Provisional application No. 60/720,248, filed on Sep. 23, 2005.

(51) Int. Cl. A61K 31/426; A61K 31/381; A61K 31/137; A61K 31/34

(52) U.S. Cl. 514/365; 514/438; 514/471; 514/649

(57) ABSTRACT

Phenylethylamine analogs useful for treating glaucoma are disclosed.

This application claims priority to U.S. Provisional Application, U.S. Ser. No. 60/720,248 filed Sep. 23, 2005.

The present invention is directed to compounds useful for treating ophthalmic diseases. In particular, the present invention is directed toward phenylethylamine analogs and their use for lowering and controlling intraocular pressure (IOP) and treating glaucoma.

### BACKGROUND OF THE INVENTION

The disease state referred to as glaucoma is characterized by a permanent loss of visual function due to irreversible damage to the optic nerve. The several morphologically or functionally distinct types of glaucoma are typically characterized by elevated IOP, which is considered to be causally related to the pathological course of the disease. Ocular hypertension is a condition wherein intraocular pressure is elevated, but no apparent loss of visual function has occurred; such patients are considered to be a high risk for the eventual development of the visual loss associated with glaucoma. Some patients with glaucomatous field loss have relatively low intraocular pressure. These so called normotension or low tension glaucoma patients can also benefit from agents that lower and control IOP. If glaucoma or ocular hypertension is detected early and treated promptly with medications that effectively reduce elevated intraocular pressure, loss of visual function or its progressive deterioration can generally be ameliorated. Drug therapies that have proven to be effective for the reduction of intraocular pressure include both agents that decrease aqueous humor production and agents that increase the outflow facility. Such therapies are in general administered by one of two possible routes, topically (direct application to the eye) or orally.

There are some individuals who do not respond well when treated with certain existing glaucoma therapies. There is, therefore, a need for other topical therapeutic agents that control IOP.

It has been found that serotonergic compounds which possess agonist activity at 5-HT₂ receptors effectively lower and control normal and elevated IOP and are useful for treating glaucoma, see U.S. Pat. No. 6,664,286. Compounds that act as agonists at 5-HT₂ receptors are well known and have shown a variety of utilities, primarily for disorders or conditions associated with the central nervous system (CNS). U.S. Pat. No. 5,494,928 discloses certain 2-(indol-1-yl)-ethylamine derivatives that are 5-HT_{2C} agonists for the treatment of obsessive compulsive disorder and other CNS derived personality disorders. U.S. Pat. No. 5,571,833 discloses tryptamine derivatives that are 5-HT₂ agonists for the treatment of portal hypertension and migraine. U.S. Pat. No. 5,874,477 discloses a method for treating malaria using 5-HT_{2A/2C} agonists. U.S. Pat. No. 5,902,815 discloses the use of 5-HT_{2A} agonists to prevent adverse effects of NMDA receptor hypo-function. WO98/31354A2 discloses 5-HT_{2B} agonists for the treatment of depression and other CNS conditions. Agonist response at the 5-HT_{2A} receptor is reported to be the primary activity responsible for hallucinogenic activity, with some lesser involvement of the 5-HT_{2C} receptor possible [Psychopharmacology, Vol. 121:357, 1995].

### SUMMARY OF THE INVENTION

The present invention is directed toward certain phenylethylamine analogs that can be used to lower and control IOP and treat glaucoma in warm blooded animals, including man. The compounds are preferably formulated in pharmaceutical compositions suitable for topical delivery to the eye.

### DESCRIPTION OF THE PREFERRED EMBODIMENTS

Compounds that are useful for lowering and controlling normal or elevated IOP and treating glaucoma according to the present invention are represented by the following formula: 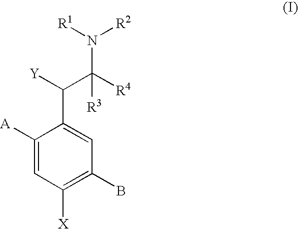
wherein
R¹ and R² independently are H or C₁₋₄alkyl;
R³ and R⁴ independently are H, C₁₋₄ alkyl, or R³, R⁴ and the carbon atom to which they are attached can form a cyclopropyl ring;
Y is H, C₁₋₄alkyl, or OR⁵;
R⁵ is H or C₁₋₄ alkyl;
A and B independently are OH, C₁₋₃alkoxy, OS(O)₂W, OC(═O)W, or OC(═O)NW²W³;
W is C₁₋₄alkyl;
W² and W³ independently are H or C₁₋₄alkyl;
X is CH(OH)R⁶, C(═O)R⁶, (CH₂)ₙZ, or (CH₂)ₘZ′;
R⁶ is C₁₋₄ alkyl, (CH₂)ₘ₂SR⁸, or (CH₂)ₘ₂OR⁸;
n is 1-4;
m is 2-4;
m² is 1-4;
Z is OCF₃, CN, S(O)ₙ₂R⁶, C(O)NR⁷R⁸, C(C═O)R⁶, CH(OH)R⁶, SO₂NR⁷R⁸, NR⁶R⁹, CO2R⁶, S(O)ₙ₂CH₂CH(OH)R⁶, S(O)ₙ₂CH₂C(═)O)R⁶, S(O)ₙ₂CH₂CO2R⁶, S(O)ₙ₂CH₂C(═O)NR⁷R⁸, aryl, heterocyclyl, or heteroaryl;
Z′ is OH or OR⁶;
n is 0 or 1;
R⁷ is H, C₁₋₄alkyl, OH, or OCH₃; and
R⁸ and R⁹ independently are H or C₁₋₄alkyl.

It is recognized that compounds of Formula (I) can contain one or more chiral centers. This invention contemplates all enantiomers, diastereomers, and mixtures thereof. In the above definitions, the total number of carbon atoms in a substituent group is indicated by the Cᵢ-Cⱼ prefix, where the numbers i and j define the number of carbon atoms; this definition includes straight chain, branched chain, and cyclic alkyl or (cyclic alkyl)alkyl groups.

The term “aryl” refers to a monocyclic, bicyclic or tricyclic ring system having a total of five to fourteen ring members, wherein at least one ring in the system is aromatic and wherein each ring in the system contains 3 to 7 ring members. The term “aryl” may be used interchangeably with the term “aryl ring”.

The term “heterocycle”, “heterocyclyl”, or “heterocyclic” as used herein means non-aromatic, monocyclic, bicyclic or tricyclic ring systems having three to fourteen ring members in which one or more ring members is a heteroatom, wherein each ring in the system contains 3 to 7 ring members.

The term “heteroaryl” refers to monocyclic, bicyclic or tricyclic ring systems having three to fourteen ring members wherein at least one ring in the system is aromatic, at least one ring in the system contains one or more heteroatoms, and wherein each ring in the system contains 3 to 7 ring members.

The term “heteroatom” means nitrogen, oxygen, or sulfur and includes any oxidized form of nitrogen and sulfur, and the quarternized form of any basic nitrogen. Also the term “nitrogen” includes a substitutable nitrogen of a heterocyclic ring. As an example, in a saturated or partially unsaturated ring having 0-3 heteroatoms selected form oxygen, sulfur or nitrogen, the nitrogen may be N (as in 3,4-dihydro-2H-pyrrolyl), NH (as in pyrrolidinyl) or NR (as in substituted pyrrolidinyl).

It is important to recognize that a substituent may be present either singly or multiply when incorporated into the indicated structural unit.

Preferred compounds of formula (I) are those in which:
R¹, R² are H;
R³ and R⁴ are independently H or C₁alkyl;
Y is H or OR⁵;
R⁵ is H or C₁alkyl;
A and B are independently OH, C₁alkoxy, or OC(═O)W;
W is C₁₋₄alkyl;
X is (CH₂)ₙZ or (CH₂)ₘZ′;
n is 1-4;
m is 2-4;
Z is OR⁶, OCF₃, S(O)ₙ₂R⁶, aryl, heterocyclyl or heteroaryl;
Z′ is OR⁶;
R⁶ is C₁₋₄ alkyl, (CH₂)ₘSR, or (CH₂)ₘOR;
heterocyclyl or heteroaryl is: 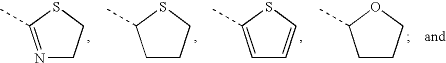
n² is 0.

The most preferred compounds of formula (I) are:

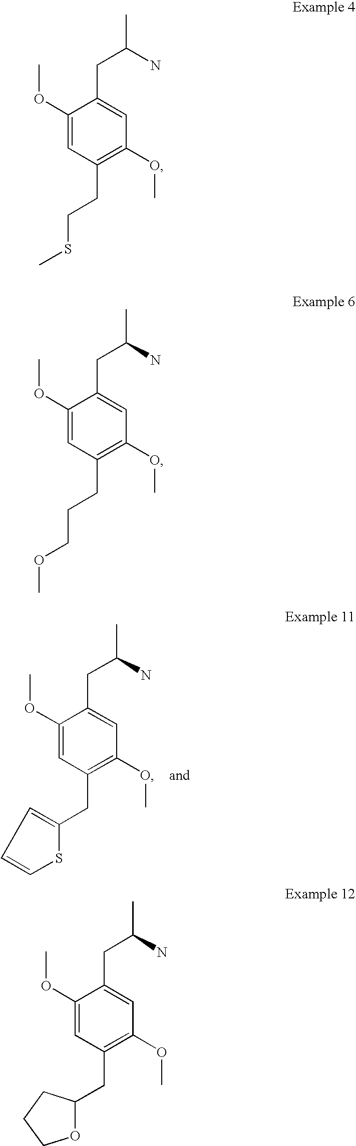

### Synthesis

The compounds of this invention can be readily prepared according to a variety of synthetic methods familiar to one skilled in the art. Representative methods are outlined in the schemes below.

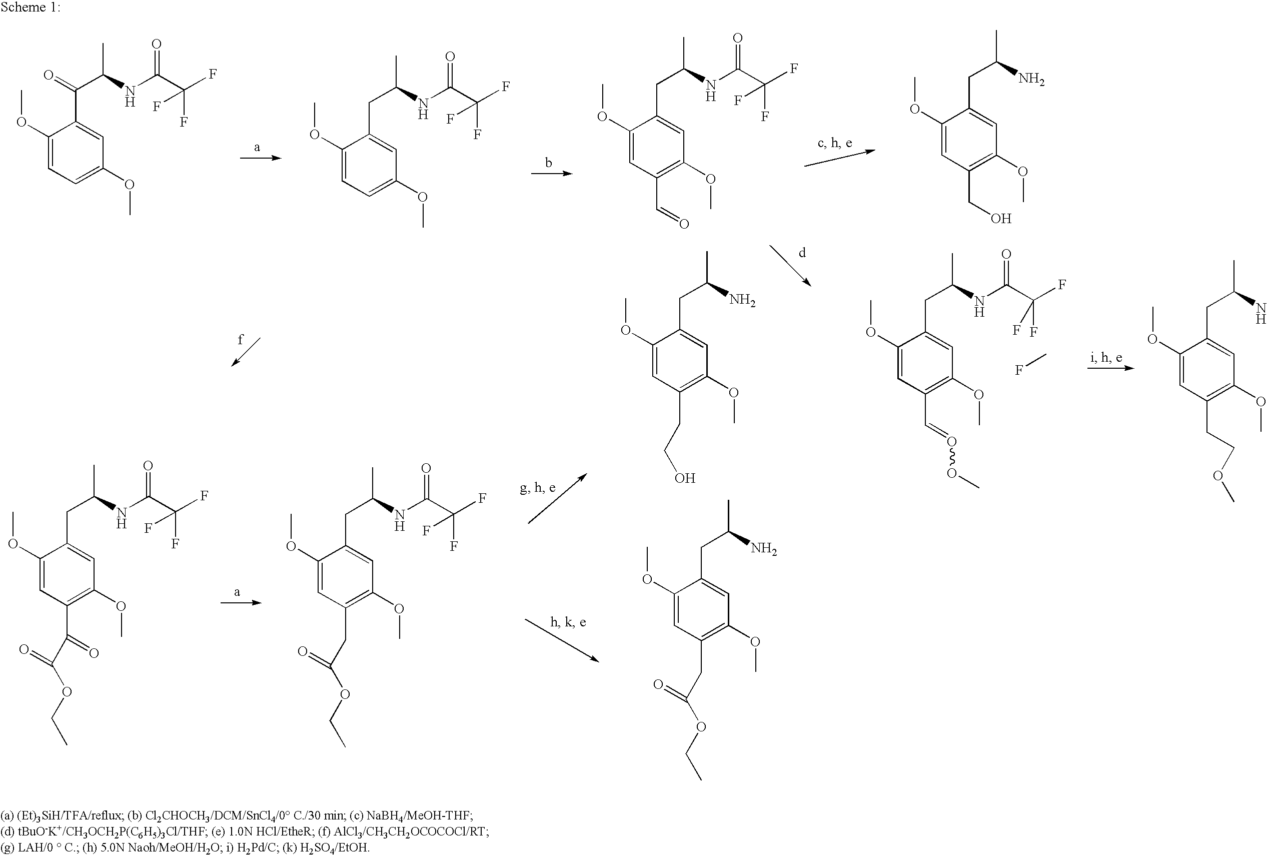

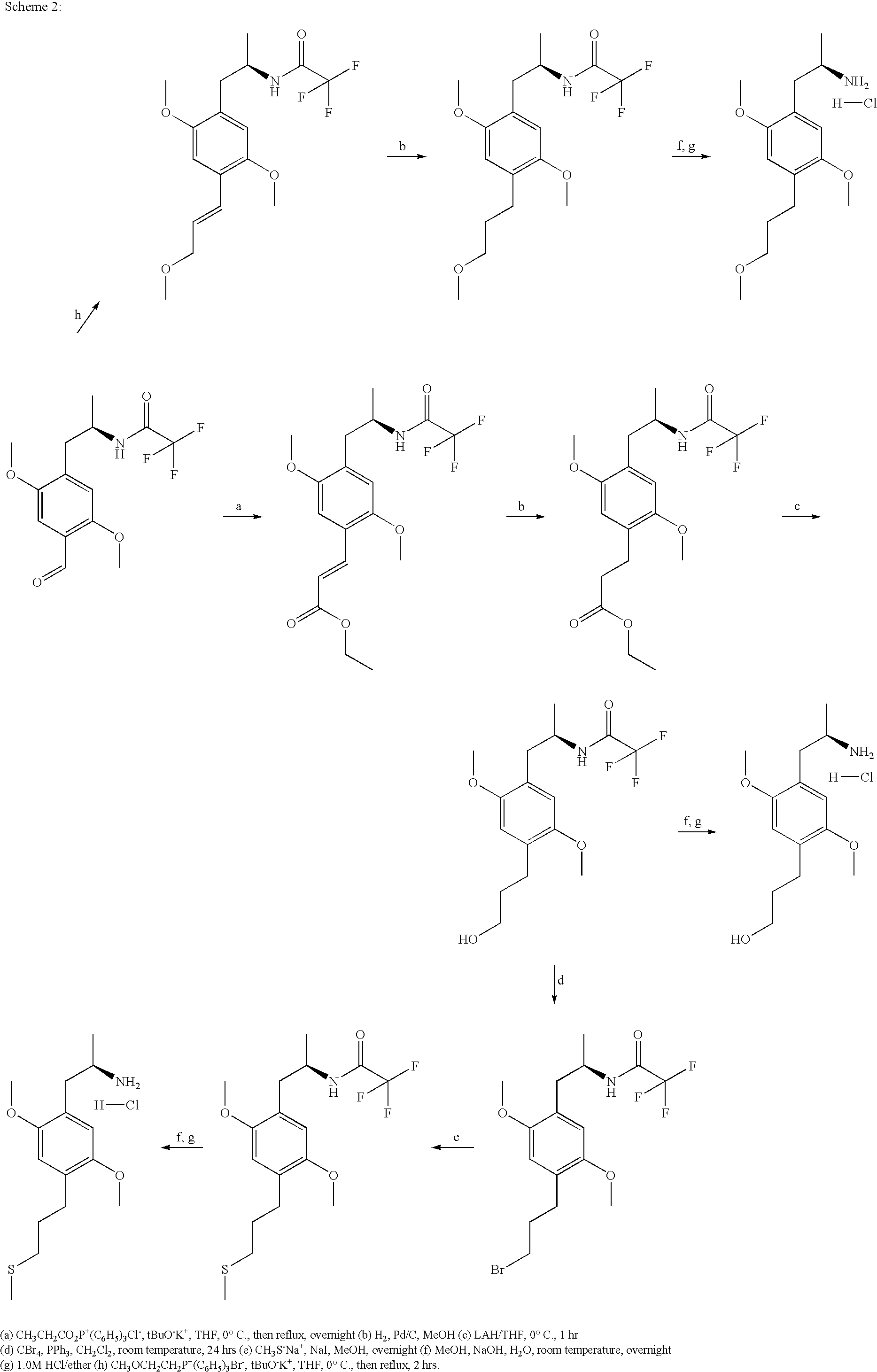

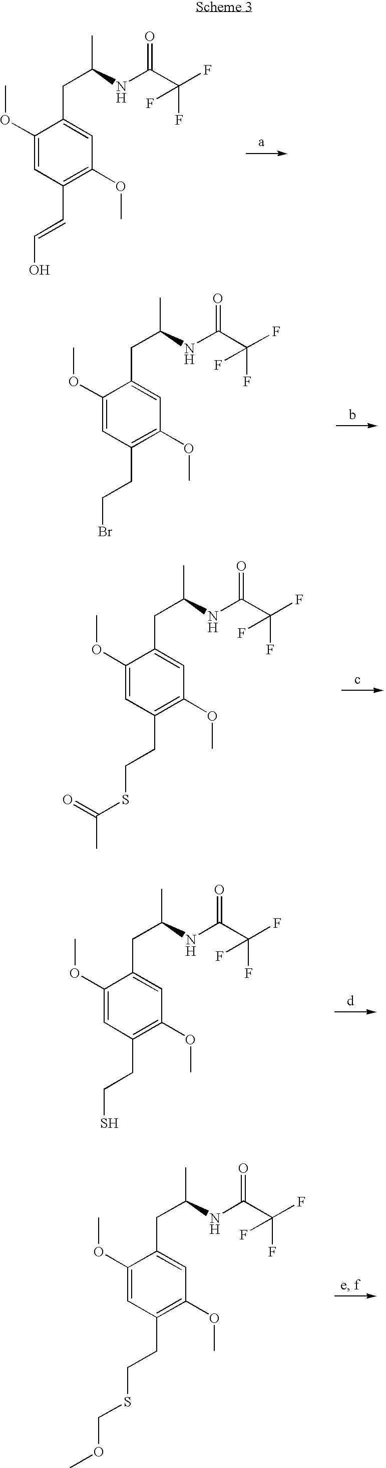

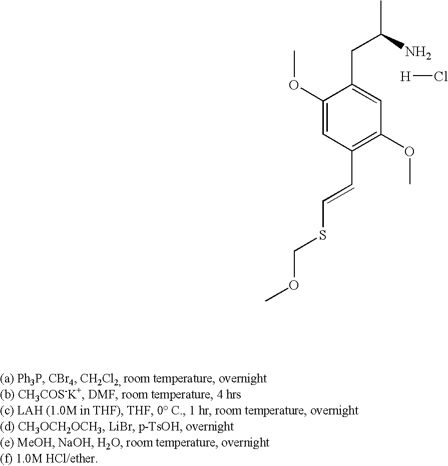

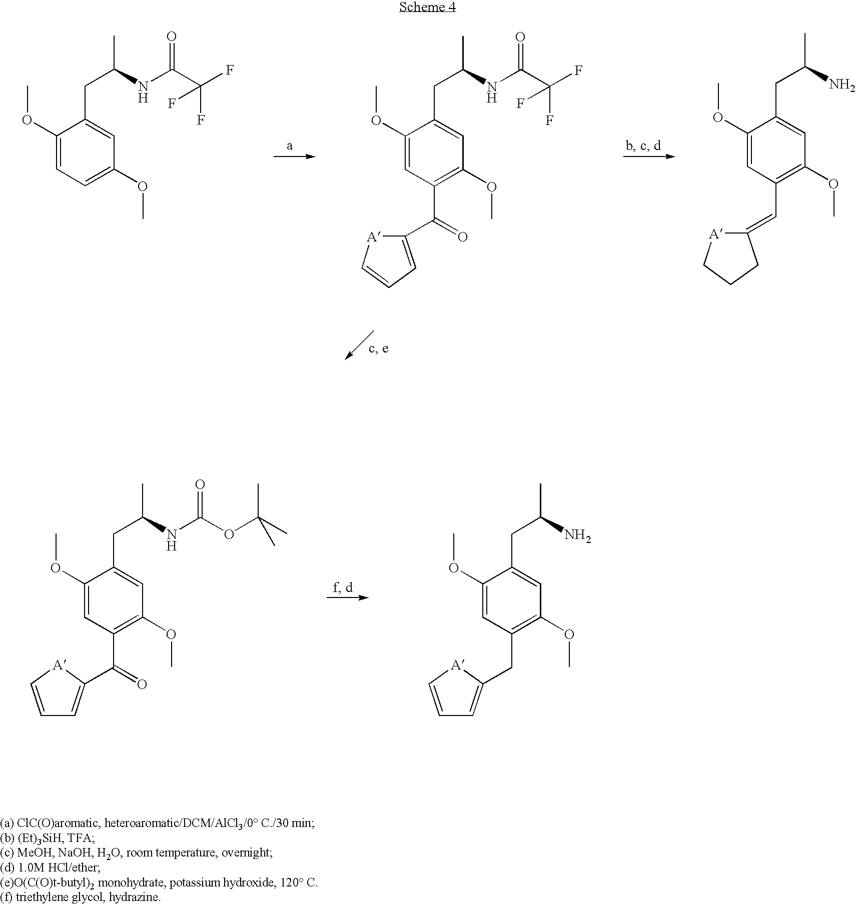

The following abbreviations have been used:
DCM dichloromethane
DIBAL diisobutylaluminum hydride
DMF dimethylformamide
EtOH ethanol
LAH lithium aluminum hydride
PPBA 3-chloroperoxybenzoic acid
TEA triethylamine

The following are general methods used to cleave 2,2,2-trifluoroacetamide protecting groups and form hydrochloride salts.

Method A: Hydrolysis of Trifluoroacetyl Groups:

The trifluoro acetamide analogs were dissolved in methanol (15 mL) and 5.0 N NaOH aqueous solution (10 mL). The reaction mixture was stirred overnight. Methanol was removed under reduced pressure, and the organic residue was extracted in dichloromethane

Method B: Hydrochloride Salts Formation:

To the free base dissolved in either diethyl ether or ethanol, is added dropwise 1.0 N HCl solution in ethyl ether. The solid formed was collected by filtration and analyzed. Recrystallization may be needed for purification. In cases where a solid did not form, the volatiles were evaporated to yield a solid.

### EXAMPLE 1

### [(R)-4-(2-Amino-propyl)-2,5-dimethoxy-phenyl]-acetic acid ethyl ester hydrochloride (Compound 1)

[(R)-4-(2-Amino-propyl)-2,5-dimethoxy-phenyl]-acetic acid ethyl ester hydrochloride was prepared by the multiple step synthetic procedure described below.

N-[(R)-2-(2,5-Dimethoxy-phenyl)-1-methyl-2-oxo-ethyl]-2,2,2-trifluoro-acetamide

To a solution of 1,4-Dimethoxybenzene (10 g, 72 mmol) and aluminum chloride (19.0 g, 145 mmol) in 200 mL of dichloromethane was added dropwise a solution of (R)-2-(2,2,2-Trifluoro-acetylamino)-propionyl chloride (29.4 g, 145 mmol) in 100 mL of dichloromethane (DCM). The reaction mixture was stirred for 6 h, and then carefully poured onto ice. This mixture was stirred until the two phases separated. The phases were then separated, and aqueous phase was extracted with an additional 200 mL of DCM. The combined organic phase was dried (anhydrous MgSO₄), and concentrated under reduced pressure. The residue was filtered through silica to give 12 g of the title compound. ¹H NMR (CDCl₃): δ ppm 1.44 (d, 3H), 3.81 (s, 3H), 3.92 (s, 3H), 5.63 (m, 1H), 6.96 (d, 1H), 7.22 (d, 1H), 7.26 (s, 1H), 7.36 (bs, 1H). LCMS (+APCI) m/z 306 (M+H).

N-[(R)-2-(2,5-Dimethoxy-phenyl)-1-methyl-ethyl]-2,2,2-trifluoro-acetamide

N-[(R)-2-(2,5-Dimethoxy-phenyl)-1-methyl-2-oxo-ethyl]-2,2,2-trifluoro-acetamide was dissolved in 50 mL of trifluoracetic acid at room temperature, and triethylsilane (18.3 g, 0.16 mol) was added dropwise. The reaction mixture was warmed at reflux for 4 h, at which time TLC showed all starting material was consumed. The reaction mixture was then allowed to cool to room temperature and poured into cold water. The titled compound precipitated and was collected by filtration. ¹H NMR (DMSO, d₆): δ ppm 1.25 (d, 3H), 2.82 (dd, 2H), 3.76 (s, 3H), 3.82 (s, 3H), 4.14 (m, 1H), 6.69-6.83 (m, 3H), 7.45 (bm, 1H). LCMS (+APCI) m/z 292 (M+H) and 309 (M+NH₄⁺)

{2,5-Dimethoxy-4-[(R)-2-(2,2,2-trifluoro-acetylamino)-propyl]-phenyl}-oxo-acetic acid ethyl ester

N-{(R)-2-[2,5-Dimethoxy-4-(2-methoxy-ethyl)-phenyl]-1-methyl-ethyl}-2,2,2-trifluoro-acetamide was prepared by the same method used to prepare Compound 1 using N-[(R)-2-(2,5-dimethoxy-phenyl)-1-methyl-ethyl]-2,2,2-trifluoro-acetamide and chloro-oxo-acetic acid ethyl ester to give the title compound in 60% yield. ¹H NMR (CDCl₃): δ ppm 1.29 (d, 3H), 1.39 (t, 3H), 2.92 (m, 2H), 3.82 (s, 3H), 3.87 (s, 3H), 4.20 (m, 1H), 4.38 (m, 2H), 6.79 (s, 1H), 7.05 (1H, NH), 6.38 (s, 1H). LCMS (+APCI) m/z 392 (M+H)

{2,5-Dimethoxy-4-[(R)-2-(2,2,2-trifluoro-acetylamino)-propyl]-phenyl}-acetic acid ethyl ester

This material was prepared in 71% yield by the same method used to prepare N-[(R)-2-(2,5-dimethoxy-phenyl)-1-methyl-ethyl]-2,2,2-trifluoro-acetamide using {2-N-{(R)-2-[2,5-dimethoxy-4-(2-methoxy-ethyl)-phenyl]-1-methyl-ethyl}-2,2,2-trifluoro-acetamide. ¹H NMR (CDCl₃): δ ppm 1.26 (m, 6H), 2.96 (m, 2H), 3.62 (m, 2H), 3.77 (s, 3H), 3.80 (s, 3H), 4.12 (m, 1H), 4.17 m, 2H), 6.65 (s, 1H), 6.78 (s, 1H), 7.46 (1H, NH). LCMS (+APCI) m/z 378 (M+H).

[4-(2-Amino-propyl)-2,5-dimethoxy-phenyl]-acetic acid ethyl ester hydrochloride (Compound 1)

To a solution of {2,5-dimethoxy-4-[2-(2,2,2-trifluoro-acetylamino)-propyl]-phenyl}-acetic acid ethyl ester (0.50 g, 1.3 mmol) in 50 mL of a mixture of methanol and water (4:1 V/V) was added 10 mL of a 5.0 N solution of sodium hydroxide. The reaction mixture was allowed to stir for 12 h, and then concentrated under reduced pressure. The residue was dissolved in 50 mL of ethanol and acidified by addition of 5 mL of sulfuric acid. The reaction mixture was warmed at reflux for 4 h, cooled and concentrated under reduced pressure. The residue was partitioned in a mixture of water and dichloromethane, and then neutralized by addition of a saturated solution of sodium bicarbonate. The organic layer was separated, dried (anhydrous MgSO₄), and the solvent was removed under reduced pressure The residue was dissolved in diethyl ether. To this solution was added 1.0 N solution of hydrogen chloride in diethyl ether. The solid that formed was collected by filtration. ¹H NMR (DMSO, d₆): δ ppm 1.10 (d, 3H), 1.15 (m, 3H), 2.69 (dd, 1H), 2.90 (dd, 1H), 3.37 (m, 1H), 3.54 (s, 2H), 3.67 (s, 3H), 3.71 (s, 3H), 4.05 (m, 2H), 6.82 (s, 1H), 6.87 (s, 1H), 8.11 (bs, 3H). CHN analysis for C₁₅H₂₃NO₄+1.0 HCl: Calculated C 56.69, H 7.61, N 4.41; Found C 56.52, H 7.68, N 4.43

### EXAMPLE 2

### (R)-2-[2,5-Dimethoxy-4-(3-methyl-[1,2,4]oxadiazol-5-ylmethyl)-phenyl]-1-methyl-ethylamine hydrochloride (Compound 2)

To a cold suspension (ice bath) of NaH (0.15 g, 4.0 mmol) in 50 mL of THF was added N-hydroxy acetamide. The heterogeneous reaction mixture was stirred at 60° C. for 30 min, and then {2,5-dimethoxy-4-[2-(2,2,2-trifluoro-acetylamino)-propyl]-phenyl}-acetic acid ethyl ester (0.5 g, 1.33 mmol) was added. The reaction mixture was stirred at reflux temperature for an additional 3 h, and allowed to cool to room temperature and partitioned in a mixture of dichloromethane/water. The organic layer was separated, and diluted with aqueous 1 N HCl. The solution was separated, neutralized by addition of a saturated bicarbonate solution, and extracted with DCM. The organic layer was dried (anhydrous MgSO₄), and concentrated under reduced pressure. The residue was converted to the hydrochloride salt by method B, and the solid that formed was recrystallized from methanol-ether to give 0.10 g of the desired material. ¹H NMR (CD₃OD): δ ppm 1.32 (d, 3H), 2.33 (s, 3H), 2.93 (m, 2H), 3.60 (m, 1H), 3.77 (s, 3H), 3.85 (s, 3H), 4.22 (s, 2H), 6.87 (s, 1H), 7.08 (s, 1H), LCMS (+APCI) m/z 292 (M+H. CHN analysis calculated for C₁₅H₂₁N₃O₃.HCl: Calculated C 54.96, H 6.76, N 12.82, found C 54.96, H 6.75, N 12.76

### EXAMPLE 3

### (R)-2-[2,5-Dimethoxy-4-(2-methoxy-ethyl)-phenyl]-1-methyl-ethylamine hydrochloride (Compound 3)

(R)-2-[2,5-Dimethoxy-4-(2-methoxy-ethyl)-phenyl]-1-methyl-ethylamine hydrochloride was prepared by the multiple step synthetic procedure described below.

2,2,2-Trifluoro-N-[(R)-2-(4-formyl-2,5-dimethoxy-phenyl)-1-methyl-ethyl]-acetamide

To a cold solution (ice bath) of N-[(R)-2-(2,5-dimethoxy-phenyl)-1-methyl-ethyl]-2,2,2-trifluoro-acetamide (0.50 g, 1.7 mmol) in 20 mL of dichloromethane were successively added dichloromethoxymethane (0.24 g, 2.1 mmol) and tin(VI) chloride (0.57 g, 2.2 mmol). The reaction mixture was stirred for 30 min, partitioned in 40 mL of water and DCM (V/V). The organic layer was separated, dried (anhydrous MgSO₄), concentrated under reduced pressure. The residue was purified by flash chromatography (SiO₂, hexanes-ethyl acetate gradient) to give 0.35 g (65%) of a slightly greenish solid. ¹H NMR (CDCl₃): δ ppm 1.31 (d, 3H), 2.84-3.00 (dd, 2H), 3.87 (s, 3H), 3.89 (s, 3H), 4.25 (m, 1H), 6.80 (s, 1H), 7.20 (m, 1H), 7.33 (bs, 1H), 10.49 (s, 1H). LCMS (+APCI) m/z 320 (M+H)

N-{(R)-2-[2,5-Dimethoxy-(4-(2-methoxy-vinyl)-phenyl]-1-methyl-ethyl}-2,2,2-trifluoro-acetamide

Methoxymethyltriphenylphosphonium chloride (1.69 g, 4.93 mmol) was placed in a 250 mL round bottom flask. Next, THF (20 mL) was added and the reaction flask was placed in an ice bath. Potassium tert-butoxide (4.9 mL, 1.0 M in THF) was added via syringe. The reaction mixture was stirred for 10 minutes. Next, compound 3 (0.75 g, 2.35 mmol), in 15 mL THF, was added via addition funnel. The reaction was stirred at 0° C. for 2 hours at which time ice bath was removed and the reaction stirred for 30 minutes at room temperature. H₂O was then added to the reaction flask. The product was extracted with EtOAc, dried with MgSO₄, and concentrated. Purification was performed using flash chromatography (SiO₂, hexanes-ethyl acetate 10%) to give a white powder: 0.63 g (1.8 mmol), 77% (a mixture of cis and trans isomers). MS (+APCI) m/z 348 (M+H).

N-{(R)-2-[2,5-Dimethoxy-4-(2-methoxy-ethyl)-phenyl]-1-methyl-ethyl}-2,2,2-trifluoro-acetamide

A solution of N-{(R)-2-[2,5-Dimethoxy-4-(2-methoxy-vinyl)-phenyl]-1-methyl-ethyl}-2,2,2-trifluoro-acetamide (0.60 g, 1.7 mmol) in 40 mL of ethanol was added to a flask that contained Pd/C (5 wt. %, 0.06 g) in ethanol (60 mL). a hydrogen balloon was affixed to reaction flask. After stirring overnight, Pd/C was removed by vacuum filtration through Celite®. Solvent was removed under reduced pressure to yield N-{(R)-2-[2,5-Dimethoxy-4-(2-methoxy-ethyl)-phenyl]-1-methyl-ethyl}-2,2,2-trifluoro-acetamide as white solid (0.54 g, 89%). ¹H NMR (CDCl₃): δ ppm 1.27 (d, 3H), 2.77-2.89 (m, 4H), 3.36 (s, 3H), 3.56-3.58 (m, 2H), 3.77 (s, 3H), 3.82 (s, 3H), 4.1 (m, 1H), 6.61 (s, 1H), 6.76 (s, 1H), 7.5 (s, 1H); LCMS (+APCI) m/z 350 (M+H).

(R)-2-[2,5-Dimethoxy-4-(2-methoxy-ethyl)-phenyl]-1-methyl-ethylamine hydrochloride (Compound 3)

N-{(R)-2-[2,5-Dimethoxy-4-(2-methoxy-ethyl)-phenyl]-1-methyl-ethyl}-2,2,2-trifluoro-acetamide (0.40 g, 1.14 mmol) prepared above was dissolved in methanol (25 mL) and a 5.0 N solution of sodium hydroxide (15 mL) was added. The solution was stirred overnight at room temperature. Solvent was removed under reduced pressure and product was extracted with dichloromethane, dried (MgSO₄), and concentrated to yield a white solid which was dissolved in anhydrous ethyl ether the free base was transformed to its hydrochloride correspondent (method B) to yield a white solid (0.23 g, 70%). ¹H NMR (DMSO, d₆): δ ppm 1.12 (d, 3H), 2.68-2.71 (m, 1H), 2.77-2.79 (m, 2H), 2.87-2.90 (m, 1H), 3.25 (s, 3H), 3.4 (m, 1H), 3.47-3.49 (m, 2H), 3.74 (m, 6H), 6.80 (s, 1H), 6.87 (s, 1H), 7.98 (s, 3H); LCMS (+APCI) m/z 254 (M+H). CHN analysis for C₁₄H₂₃NO₃+1HCl: Calculated: C 58.02, H 8.35, N 4.83; Found: C 58.21, H 8.37, N 4.83.

### EXAMPLE 4

### 2-[2,5-Dimethoxy-4-(2-methylsulfanyl-ethyl)-phenyl]-1-methyl-ethylamine hydrochloride (Compound 4)

2-[2,5-Dimethoxy-4-(2-methylsulfanyl-ethyl)-phenyl]-1-methyl-ethylamine hydrochloride (Compound 4) was prepared by a multiple step synthetic procedure.

Bromo-4-(2-bromo-ethyl)-2,5-dimethoxy-benzene

To a cold solution of 2-(4-bromo-2,5-dimethoxy-phenyl)-ethanol, (Waldman, Steve R.; Monte, Aaron P.; Bracey, Ann; Nichols, David E.; Tetrahedron Lett.; EN; 37; 43; 1996; 7889-7892), (19.67 g, 75.36 mmol) in 200 mL of DCM was successively added CBr₄ (27.49 g, 82.90) and PPh₃ (21.74 g, 82.90). The reaction mixture was stirred for 1 h, allowed to warm to room temperature and stirred an additional 4 h. After the reaction mixture was concentrated under reduced pressure, the residue was purified by flash chromatography (SiO₂, hexanes-ethyl acetate gradient) to give 15.00 g of bromo-4-(2-bromo-ethyl)-2,5-dimethoxy-benzene as a white solid. ¹H NMR (CDCl₃): δ ppm 3.12 (m, 2H), 3.55 (m, 2H), 3.79 (s, 3H), 3.85 (s, 3H), 6.75 (s, 1H), 7.04 (s, 1H). LCMS (+Cl) m/z 322, 323, 324, 325, 326, 327.

1-Bromo-2,5-dimethoxy-4-(2-methylsulfanyl-ethyl)-benzene

To a solution of bromo-4-(2-bromo-ethyl)-2,5-dimethoxy-benzene (1.5 g, 4.6 mmol) in ethanol (60 mL) was added sodium thiomethoxide (0.65 g, 9.2 mmol), and potassium iodide (0.76 g, 4.6 mmol). The reaction mixture was refluxed for 3 h, and then cooled to room temperature. The solvent was removed under reduced pressure and the residue was purified by flash chromatography (SiO₂, hexanes-ethyl acetate gradient) to give 0.80 g of a white solid. ¹H NMR (CDCl₃): δ ppm 2.06 (s, 3H), 2.63 (m, 2H), 2.78 (m, 2H), 3.71 (s, 3H), 3.77 (s, 3H), 6.68 (s, 1H), 6.95 (s, 1H).

2,5-Dimethoxy-4-(2-methylsulfanyl-ethyl)-benzaldehyde

A solution of 1-bromo-2,5-dimethoxy-4-(2-methylsulfanyl-ethyl)-benzene (2.5 g, 8.62 mmol) in 100 mL of THF was cooled to −78° C. (dry ice-acetone bath) and stirred for 10 min. To this solution was added 3.6 mL of 2.5 M solution of n-BuLi in hexanes via syringe. The reaction mixture was allowed to stir for 10 min and then was quenched by the addition of 5 mL of DMF. The temperature of the reaction mixture was allowed to increase to room temperature and the solution was partitioned between water and ethyl acetate. The organic layer was separated, dried (anhydrous MgSO₄), and the solvent was removed under reduced pressure. The residue was purified by flash chromatography (SiO₂, hexanes-ethyl acetate 5%) to give 1.2 g of white solid. ¹H NMR (DMSO, d₆): δ ppm 2.16 (s, 3H), 2.76 (m, 2H), 2.94 (m, 2H), 3.85 (s, 3H), 3.94 (s, 3H), 7.21(s, 1H), 7.23 (s, 1H), 10.35 (s, 1H).

1,4-Dimethoxy-2-(2-methylsulfanyl-ethyl)-5-(2-nitro-propenyl)-benzene

To a solution of 2,5-dimethoxy-4-(2-methylsulfanyl-ethyl)-benzaldehyde (1.06 g, 4.44 mmol) in 50 mL of ethanol was added nitroethane (1.33 g, 17.2 mmol) and ammonium acetate (0.340 g, 4.44 mmol). The reaction mixture was warmed at refluxed overnight. Volatiles were removed under reduced pressure and the residue was added to water and extracted with ethyl acetate. The organic layer was dried (anhydrous MgSO₄), and ethyl acetate was removed under reduced pressure to give a yellow solid. ¹H NMR (DMSO, d₆): δ ppm 2.14 (s, 3H), 2.41 (s, 3H), 2.54 (m, 2H), 2.92 (m, 2H), 3.83 (s, 3H), 3.86 (s, 3H), 7.02 (s, 1H), 7.07 (s, 1H), 8.17 (s, 1H).

2-[2,5-Dimethoxy-4-(2-methylsulfanyl-ethyl)-phenyl]-1-methyl-ethylamine hydrochloride (Compound 4)

The yellow solid obtained above (1.17 g, 3.93.mmol) was dissolved in THF and the resulting solution cooled down to 0° C. To this solution was added 15.8 mL of 1.0 N solution of LAH in THF. The reaction mixture was stirred overnight at room temperature. Excess LAH was destroyed by consecutive addition of 0.6 mL of water, 0.6 mL solution of 15% NaOH, and 1.8 mL of water. The solid formed was washed by DCM and removed by filtration. The filtrate was extracted with aqueous 1N HCl . The aqueous layer was separated and neutralized with a saturated solution of NaHCO₃. The mixture was then extracted with DCM. The organic layer was concentrated in vacuo. The residue was dissolved in ethyl ether and converted to the hydrochloride salt by method B. The product formed as a white solid (0.70 g). ¹H NMR (DMSO, d₆): δ ppm 1.10 (d, 3H), 2.09 (s, 3H), 2.49-2.79 (m, 6H), 3.35 (m, 1H), 3.74 (s, 3H), 3.75 (s, 3H), 6.81 (s, 1H), 6.88 (s, 1H), 8.03 (bs, 3H). LCMS (+APCI) m/z 270 (M+H). CHN analysis calculated for C₁₄H₂₃NO₂S+1.0 HCl: Calculated C 54.98, H 7.91, N 4.58, found C 54.74, H 7.89, N 4.47

### EXAMPLE 5

### (R)-2-[2,5-Dimethoxy-4-(3-methylsulfanyl-propyl)-phenyl]-1-methyl-ethylamine hydrochloride (Compound 5)

(R)-2-[2,5-Dimethoxy-4-(3-methylsulfanyl-propyl)-phenyl]-1-methyl-ethylamine; hydrochloride (Compound 5) was prepared by a multiple step synthetic procedure.

3-{2,5-Dimethoxy-4-[(R)-2-(2,2,2-trifluoro-acetylamino)-propyl]-phenyl}-acrylic acid ethyl ester

(Ethoxycarbonyl methyl)-triphenyl phosphonium chloride 80% tech. grade (3.86 g, 8.02 mmol) was dispersed in THF (40 mL) and cooled to 0° C. After ten minutes, potassium tert-butoxide 1.0 M in THF (8.0 mL) was added via syringe. Next, 2,2,2-Trifluoro-N-[(R)-2-(4-formyl-2,5-dimethoxy-phenyl)-1-methyl-ethyl]-acetamide (0.64 g, 2.00 mmol) in 10 mL THF was added via addition funnel. The Ice bath was removed and the reaction was heated at gentle reflux overnight. Product mixture was concentrated. Purification was performed using flash chromatography (10% EtOAc in Hexanes) to yield a white solid: 0.60 g (77%). MS (APCI) m/z 390 (M+H).

3-{2,5-Dimethoxy-4-[(R)-2-(2,2,2-trifluoro-acetylamino)-propyl]-phenyl}-propionic acid ethyl ester

3-{2,5-Dimethoxy-4-[(R)-2-(2,2,2-trifluoro-acetylamino)-propyl]-phenyl}-acrylic acid ethyl ester (0.60 g, 1.5 mmol), in ethanol (10 mL) was treated with 5% Pd/C and stirred under an atmosphere of hydrogen overnight. The product mixture was filtered through Celite®, concentrated, and purified by flash chromatography (10% EtOAc in Hexanes) to give a white solid: 0.57 g (1.5 mmol), 95%. MS (APCI) m/z 392 (M+H).

2,2,2-Trifluoro-N-((R)-2-[4-(3-hydroxy-propyl)-2,5-dimethoxy-phenyl]-1-methyl-ethyl}-acetamide

3-{2,5-Dimethoxy-4-[(R)-2-(2,2,2-trifluoro-acetylamino)-propyl]-phenyl}-propionic acid ethyl ester (0.57 g, 1.46 mmol) was dissolved in THF (35 mL) and cooled to 0° C. LAH 1.0 M in THF (5.8 mL) was slowly addition via addition funnel. The reaction was stirred for 1 hour at 0° C. THF was evaporated. The product was dissolved in EtOAc, washed with saturated NaCl solution, dried with MgSO₄, and concentrated to yield a white solid: 0.40 g (79%). MS (APCI) m/z 350 (M+H).

N-{(R)-2-[4-(3-Bromo-propyl)-2,5-dimethoxy-phenyl]-1-methyl-ethyl}-2,2,2-trifluoro-acetamide

2,2,2-Trifluoro-N-{(R)-2-[4-(3-hydroxy-propyl)-2,5-dimethoxy-phenyl]-1-methyl-ethyl}-acetamide (0.72 g, 2.1 mmol) was dissolved in DCM (50 mL). Triphenylphosphine (0.57 g, 2.2 mmol) and carbon tetrabromide (0.72 g, 2.16 mmol) were added. The reaction was stirred at room temperature for 24 hours. Ethanol was added and reaction stirred for an additional 2 hours. The volatiles were evaporated. Purification was performed using flash chromatography (10% EtOAc in Hexanes) to give a white solid: 0.67 g (79%). MS (APCI) m/z 412 (M+H).

N-{(R)-2-[2,5-Dimethoxy-4-(3-methylsulfanyl-propyl)-phenyl]-1-methyl-ethyl}-2,2,2-trifluoro-acetamide

N-{(R)-2-[4-(3-Bromo-propyl)-2,5-dimethoxy-phenyl]-1-methyl-ethyl}-2,2,2-trifluoro-acetamide (0.26 g, 0.63 mmol), sodium iodide (0.10 g, 0.69 mmol), and sodium thiomethoxide in methanol (15 mL) were warmed at reflux for approximately 18 hours. The volatiles were removed under reduced pressure. The residue was dissolved in ethyl acetate, washed with water and saturated NaCl solution, dried with anhydrous MgSO₄, and concentrated to yield the title compound as an off-white solid: 0.20 g (84%). MS (APCI) m/z 380 (M+H).

(R)-2-[2,5-Dimethoxy-4-(3-methylsulfanyl-propyl)-phenyl]-1-methyl-ethylamine; hydrochloride (Compound 5)

N-{(R)-2-[2,5-Dimethoxy-4-(3-methylsulfanyl-propyl)-phenyl]-1-methyl-ethyl}-2,2,2-trifluoro-acetamide (0.10 g, 0.40 mmol) was dissolved in methanol (20 mL). Next, a 15% NaOH solution (15 mL) was added. The reaction was stirred at room temperature overnight. Solvent was removed and product was extracted with DCM. The organic extracts were dried with anhydrous MgSO₄, and concentrated to yield an off-white solid which was dissolved in anhydrous ethyl ether. To this solution was added 1.0M solution of hydrogen chloride in ethyl ether. The precipitate was collected by vacuum filtration to give a tan solid: 0.041 g (55%). ¹H NMR (600 MHz, DMSO, d₆): δ ppm 1.12 (m, 3H), 1.76-1.80 (m, 2H), 2.05 (s, 3H), 2.46-2.48 (m, 2H), 2.60-2.62 (m, 2H), 2.64 (m, 1H), 2.70 (m, 1H), 3.4 (m, 1H), 3.74 (s, 6H), 6.80-6.82 (m, 2H), 7.99 (bs, 3H). MS (APCI) m/z 284 (M+H). CHN analysis for C₁₅H₂₅NO₂.1.0 HCl+0.6 H₂O: Calculated C 54.48, H 8.29, N 4.24; Found C 54.46, H 8.02, N 4.17.

### EXAMPLE 6

### (R)-2-[2,5-Dimethoxy-4-(3-methoxy-propyl)-phenyl]-1-methyl-ethylamine hydrochloride (Compound 6)

(R)-2-[2,5-Dimethoxy-4-(3-methoxy-propyl)-phenyl]-1-methyl-ethylamine hydrochloride was prepared by a multiple step synthetic procedure.

N-{(R)-2-[2,5-Dimethoxy-4-(-3-methoxy-propenyl)-phenyl]-1-methyl-ethyl}-2,2,2-trifluoro-acetamide

(2-Methoxy-ethyl)-triphenyl-phosphonium; bromide (5.03 g, 12.5 mmol) was dispersed in THF (100 mL) and cooled to 0° C. Next, potassium tert-butoxide 1.0 M in THF (12.5 mL) was added via syringe. 2,2,2-Trifluoro-N-[(R)-2-(4-formyl-2,5-dimethoxy-phenyl)-1-methyl-ethyl]-acetamide was added. The ice bath was removed and the reaction was warmed at reflux for 2 hours. The mixture was diluted with ethyl acetate, washed with saturated NaCl solution, dried with anhydrous MgSO₄, concentrated, and purified using flash chromatography (10% ethyl acetate in hexanes) to yield a white solid: 0.55 g (44%). MS (APCI) m/z 379 (M+NH₄⁺).

N-{(R)-2-[2,5-Dimethoxy-4-(3-methoxy-propyl)-phenyl]-1-methyl-ethyl}-2,2,2-trifluoro-acetamide

N-{(R)-2-[2,5-Dimethoxy-4-(-3-methoxy-propenyl)-phenyl]-1-methyl-ethyl}-2,2,2-trifluoro-acetamide (0.52 g, 1.4 mmol), in ethanol (50 mL) was treated with 5% Pd/C and stirred under an atmosphere of hydrogen overnight. The product mixture was filtered through Celite®, concentrated, and purified by flash chromatography (10% ethyl acetate in hexanes) to give a white solid: 0.30 g (57%). MS (APCI) m/z 364 (M+H).

(R)-2-[2,5-Dimethoxy-4-(3-methoxy-propyl)-phenyl]-1-methyl-ethylamine hydrochloride (Compound 6)

N-{(R)-2-[2,5-Dimethoxy-4-(3-methoxy-propyl)-phenyl]-1-methyl-ethyl}-2,2,2-trifluoro-acetamide (0.20 g, 0.55 mmol) was dissolved in methanol (25 mL) in a 250 mL round bottom flask. Next, a 15% NaOH solution (15 mL) was added. The reaction was stirred at room temperature overnight. Solvent was removed and product was extracted with DCM, dried with anhydrous MgSO₄, and concentrated to yield a white solid which was dissolved in anhydrous ethyl ether. To this solution was added 1.0M solution of hydrogen chloride in ethyl ether. The precipitate was collected by vacuum filtration to give a white solid: 0.080 g (54%). ¹H-NMR (400 MHz, DMSO, d₆): δ ppm 1.12 (m, 3H), 1.72-1.76 (m, 2H), 2.56-2.58 (m, 2H), 2.67-2.70 (m, 1H), 2.87-2.88 (m, 1H), 3.32 (m, 2H), 3.38 (m, 1H), 3.74 (s, 6H), 6.80 (s, 2H), 8.02 (bs, 3H). MS (APCI) m/z 268 (M+H). CHN analysis for C₁₅H₂₅NO₃.HCl: Calculated: C 59.30, H 8.63, N 4.61; Found: C 59.12, H 8.57, N 4.57.

### EXAMPLE 7

### (R)-2-[2,5-Dimethoxy-4-(2-methoxymethylsulfanyl-ethyl)-phenyl]-1-methyl-ethylamine hydrochloride (Compound 7)

(R)-2-[2,5-Dimethoxy-4-(2-methoxymethylsulfanyl-ethyl)-phenyl]-1-methyl-ethylamine hydrochloride (Compound 7) was prepared by a multiple step synthetic procedure.

2,2,2-Trifluoro-N-{(R)-2-[4-(2-hydroxy-ethyl)-2,5-dimethoxy-phenyl]-1-methyl-ethyl}-acetamide

2,2,2-Trifluoro-N-{(R)-2-[4-(2-hydroxy-ethyl)-2,5-dimethoxy-phenyl]-1-methyl-ethyl}-acetamide was prepared from {2,5-dimethoxy-4-[(R)-2-(2,2,2-trifluoro-acetylamino)-propyl]-phenyl}-acetic acid ethyl ester by the same procedure as described for 2,2,2-Trifluoro-N-{(R)-2-[4-(3-hydroxy-propyl)-2,5-dimethoxy-phenyl]-1-methyl-ethyl}-acetamide. MS (APCI) m/z 336 (M+H).

N-{(R)-2-[4-(2-Bromo-ethyl)-2,5-dimethoxy-phenyl]-1-methyl-ethyl}-2,2,2-trifluoro-acetamide

2,2,2-Trifluoro-N-{(R)-2-[4-(2-hydroxy-ethyl)-2,5-dimethoxy-phenyl]-1-methyl-ethyl}-acetamide (3.60 g, 10.7 mmol) and triphenylphosphine (2.96 g, 11.3 mmol) were dissolved in DCM (150 mL). Next, carbon tetrabromide (3.74 g, 11.3 mmol) was added slowly. After stirring overnight at room temperature, volatiles were removed to yield a light yellow solid. Crude product was purified by flash chromatography on silica gel eluting with ethyl acetate:hexanes (10:90) to yield a white solid: 2.6 grams (61%). ¹H NMR (CDCl₃): δ 1.27 (m, 3H), 2.76-2.90 (m, 2H), 3.12-3.17 (m, 2H), 3.54-3.58 (m, 2H), 3.78 (s, 3H), 3.82 (s, 3H), 4.1 (m, 1H), 6.62 (s, 1H), 6.72 (s, 1H), 7.3 (bs, 1H). MS (APCI) m/z 398 (M+H).

Thioacetic acid S-(2-{2,5-dimethoxy-4-[(R)-2-(2,2,2-trifluoro-acetylamino)-propyl]-phenyl}-ethyl) ester

N-{(R)-2-[4-(2-Bromo-ethyl)-2,5-dimethoxy-phenyl]-1-methyl-ethyl}-2,2,2-trifluoro-acetamide (2.5 g, 6.3 mmol) and potassium thioacetate (0.81 g, 7.1 mmol) were dissolved in DMF (80 mL). After stirring at room temperature under nitrogen for 4 hours, volatiles were removed. The product was extracted with ethyl acetate, washed with saturated NaCl solution, dried with anhydrous MgSO₄, and concentrated to yield a light brown solid. The crude product was purified by flash chromatography on silica gel eluting with ethyl acetate:hexanes (10:90) to yield an off-white solid: 2.0 grams (80%). ¹H NMR (CDCl₃): δ 1.27 (m, 3H), 2.33 (s, 3H), 2.78-2.89 (m, 4H), 3.09-3.12 (m, 2H), 3.78 (s, 3H), 3.83 (s, 3H), 4.1 (m, 1H), 6.61 (s, ₁H), 6.72 (s, 1H), 7.4 (bs, 1H). MS (APCI) m/z 394 (M+H).

2,2,2-Trifluoro-N-{(R)-2-[4-(2-mercapto-ethyl)-2,5-dimethoxy-phenyl]-1-methyl-ethyl}-acetamide

Thioacetic acid S-(2-{2,5-dimethoxy-4-[(R)-2-(2,2,2-trifluoro-acetylamino)-propyl]-phenyl}-ethyl) ester (1.82 g, 4.63 mmol) was dissolved in THF and cooled to 0° C. in ice bath. A solution of 1.0M LAH in THF (18.5 mL) was added slowly via addition funnel. Reaction was stirred at 0° C. under nitrogen for 1 hour. Water was added to quench the reaction. The product was extracted with ethyl acetate, dried with anhydrous MgSO₄, and concentrated to yield a white solid: 1.23 grams (76%). MS (APCI) m/z 352 (M+H).

N-{(R)-2-[2,5-Dimethoxy-4-(2-methoxymethylsulfanyl-ethyl)-phenyl]-1-methyl-ethyl}-2,2,2-trifluoro-acetamide

2,2,2-Trifluoro-N-{(R)-2-[4-(2-mercapto-ethyl)-2,5-dimethoxy-phenyl]-1-methyl-ethyl}-acetamide (1.2 g, 3.5 mmol), lithium bromide (0.33 g, 3.9 mmol), and p-toluenesulfonic acid (0.06 g) were combined in reaction flask. Next, dimethoxymethane (65 mL) was added. After stirring at room temperature under nitrogen for 24 hours, product was extracted with ethyl acetate, washed with water and brine, dried with anhydrous MgSO₄, and concentrated to yield an off-white solid. Crude product was purified by flash chromatography on silica gel eluting with ethyl acetate:hexanes (10:90) to yield a white solid: 1.2 grams (87%). ¹H NMR (CDCl₃): δ 1.27 (m, 3H), 2.80-2.93 (m, 6H), 3.35 (s, 3H), 3.78 (s, 3H), 3.82 (s, 3H), 4.1 (m, 1H), 4.65 (s, 2H), 6.61 (s, 1H), 6.73 (s, 1H), 7.4 (bs, 1H). MS (APCI) m/z 413 (M+NH₄⁺).

(R)-2-[2,5-Dimethoxy-4-(2-methoxymethylsulfanyl-ethyl)-phenyl]-1-methyl-ethylamine hydrochloride (Compound 7)

N-{(R)-2-[2,5-Dimethoxy-4-(2-methoxymethylsulfanyl-ethyl)-phenyl]-1-methyl-ethyl}-2,2,2-trifluoro-acetamide (0.30 g, 0.76 mmol) was dissolved in methanol (25 mL). Next, a 15% NaOH solution (15 mL) was added. The reaction was stirred at room temperature overnight. Methanol was removed and the product was extracted with DCM, dried with MgSO₄, and concentrated to yield a off-white solid which was dissolved in anhydrous ethyl ether. To this solution was added 1.0M solution of hydrogen chloride in ethyl ether dropwise until no further formation of precipitate. Ether was evaporated to yield a tan solid: 0.127 g (56%). ¹H NMR (400 MHz, DMSO, d₆): δ 1.12 (m, 3H), 2.67-2.82 (m, 5H), 2.89-2.93 (m, 1H), 3.4 (s, 1H), 3.75 (s, 6H), 4.67 (s, 2H), 6.81 (s, 1H), 6.87 (s, 1H), 8.09 (bs, 3H). MS (APCI) m/z 300 (M+H). CHN analysis for C₁₅H₂₅NO₃S.1.0 HCl: Calculated: C 53.64, H 7.80, N 4.17; Found: C 53.64, H 7.86, N 4.17.

### EXAMPLE 8

### Methanesulfonic acid 5-(2-amino-propyl)-4-methoxy-2-methyl-phenyl ester hydrochloride (Compound 8)

To a solution of 2-(5-Benzyloxy-2-methoxy-4-methyl-phenyl)-1-methyl-ethylamine hydrochloride, (for preparation see Zweig, J. S.; Castagnoli, N.; J. Med. Chem.; EN; 20; 1977; 414-421), (0.50 g, 1.6 mmol) in DCM (40 mL) were added TEA (2 mL), and tert-butyl dicarbonate (0.40 g, 1.9 mmol). The reaction mixture was stirred for 2 h, and diluted with 1 N HCl aqueous solution. The organic layer was separated, dried (anhydrous MgSO₄), and concentrated under reduced pressure. The residue was dissolved in methanol, and Pd/C 5% (100 mg) was then added, and stirred under a hydrogen environment overnight. The catalyst was removed by filtration on Celite® and the filtrate was concentrated to give 250 mg of [2-(5-Hydroxy-2-methoxy-4-methyl-phenyl)-1-methyl-ethyl]-carbamic acid tert-butyl ester. LCMS (+APCI) m/z 296 (M+H).

To a solution of [2-(5-Hydroxy-2-methoxy-4-methyl-phenyl)-1-methyl-ethyl]-carbamic acid tert-butyl ester (0.11 g, 0.37 mmol) in DCM (10 mL) was added TEA (0.2 mL) and methanesulfonyl chloride (0.047 g, 0.41 mmol). The reaction mixture was stirred at room temperature for 2 h, and diluted with 1 N HCl aqueous solution. The organic layer was separated, dried (anhydrous MgSO₄), and concentrated. The residue was purified by flash chromatography to give an oily residue which was dissolved in a (1/1) mixture of DCM-TFA (10 mL) and stirred for 3 h. Volatiles were evaporated under reduced pressure, the residue was partitioned between water and dichloromethane and neutralized by addition of a saturated solution of NaHCO₃. The organic layer was separated, dried (anhydrous MgSO₄), and removed under reduced pressure to give 30 mg of the free base as an oil. This was converted to the corresponding hydrochloride salt (method B). ¹H NMR (DMSO, d₆): δ ppm 1.04 (d, 3H), 2.21 (s, 3H), 2.70-2.80 (m, 2H), 3.33 (m, 4H), 3.73 (s, 3H), 6.92 (s, 1H), 7.08 (s, 1H), 7.89 (bs, 3H). LCMS (+APCI) m/z 274 (M+H).

### EXAMPLE 9

### Acetic acid 5-(2-Amino-propyl)-4-methoxy-2-methyl-phenyl ester hydrochloride (Compound 9)

Compound 9 was prepared by the same procedure used for example 8, using acetic anhydride as the acylating reagent. ¹H NMR (DMSO, d₆): δ ppm 1.08 (d, 3H), 2.10 (s, 3H), 2.27 (s, 3H), 2.70-2.90 (m, 2H), 3.33 (m, 1H), 3.79 (s, 3H), 6.87 (s, 1H), 6.92 (s, 1H), 7.98 (bs, 3H). LCMS (+APCI) m/z 238 (M+H).

### EXAMPLE 10

### (R)-2-[4-(4,5-Dihydro-thiazol-2-ylmethyl)-2,5-dimethoxy-phenyl]-1-methyl-ethylamine hydrochloride (Compound 10)

To a solution of cysteamine hydrochloride (0.8 g, 7.04 mmol) in toluene (60 mL) was added 1.0 M solution of i-Bu3Al (19.89 mL, 19.89 mmol) in toluene under nitrogen. The mixture was stirred for at reflux for 1 h. To this solution was added {2,5-dimethoxy-4-[2-(2,2,2-trifluoro-acetylamino)-propyl]-phenyl}-acetic acid ethyl ester (3.0 g, 7.95 mmol), and the mixture was warmed at reflux for 2 h, cooled to room temperature, quenched by dropwise addition of 5 mL of methanol, and stirred for 10 min. To this solution was added 20 mL of saturated Rochelle's salts solution, 20 ml of saturated solution of Na₂CO₃, and 20 mL of saturated NaCl. To this solution was added ethyl acetate (100 mL), and the mixture was stirred vigorously for 15 min. The organic layer was separated, dried (anhydrous MgSO₄), and the solvent was removed under reduced pressure. The residue was hydrolyzed by method A, and the free base was converted to its hydrochloride salt by method B. Recrystallization in methanol-diethyl ether gave 240 mg of an off white solid. ¹H NMR (DMSO, d₆): δ 1.13 (d, 3H), 2.71-2.96 (dd, 2H), 3.5 (m, 1H), 3.57 (m, 2H), 3.75-377 (2s, 6H), 4.06 (s, 2H), 4.28 (m, 2H), 6.93 (s, 1H), 7.01 (s, 1H), 8.14 (bs, 3H). LCMS (+APCI) m/z 295 (M+H). CHN analysis calculated for C₁₅H₂₂N₂O₂S+2.0 HCl+0.6 H₂O: Calculated C 47.64, H 6.72, N 7.41, found C 47.51, H 6.50, N 7.33

### EXAMPLE 11

### (R)-2-(4-Furan-2-ylmethyl-2,5-dimethoxy-phenyl)-1-methyl-ethylamine hydrochloride (Compound 11)

(R)-N-{2-[2,5-Dimethoxy-4-(thiophene-2-carbonyl)-phenyl]-1-methyl-ethyl}-2,2,2-trifluoro-acetamide was hydrolyzed by method A and protected with the tert-butoxycarbonyl anhydride to give {2-[2,5-dimethoxy-4-(thiophene-2-carbonyl)-phenyl]-1-methyl-ethyl}-carbamic acid tert-butyl ester in 49% overall yield. ¹H NMR (400 MHz, CDCl₃): δ 1.17 (d, 3H), 1.40 (s, 9H), 2.79 (m, 2H), 3.74 (m, 3H), 3.79 (m, 3H), 3.83 (m, 1H), 4.7 (bs, 1H), 6.82 (s, 1H), 6.92 (s, 1H), 7.10 (m, 1H), 7.50 (m, 1H), 7.68 (m, 1H). This compound (1.0 g, 2.46 mmol) was dissolved in triethylene glycol (5 mL), hydrazine monohydrate (0.48 g, 9.85 mmol) was added followed by potassium hydroxide (0.54 g, 9.85 mmol). The reaction mixture was heated at 120° C. overnight, allowed to cool to room temperature and diluted with DCM (50 mL) and water (50 mL). The organic layer was separated, dried (anhydrous MgSO₄), and the volatiles were removed in under reduced pressure to give the desired material. This was transformed to hydrochloride salt by method B, and purified by crystallization to give 170 mg of the title compound. ¹H NMR (400 MHz, DMSO, d₆): δ 1.12 (d, 3H), 2.71-2.89 (m, 2H), 3.41 (m, 1H), 3.71 (m, 3H), 3.74 (m, 3H), 4.06 (s, 2H), 6.88-6.92 (m, 4H), 7.28 (d, 1H), 8.03 (bs, 3H). MS (APCI) m/z 292 (M+H).

### EXAMPLE 12

### (R)-2-[2,5-Dimethoxy-4-(tetrahydro-furan-2-ylmethyl)-phenyl]-1-methyl-ethylamine hydrochloride (Compound 12)

The title compound was prepared by the same procedure reported for Example 1 using N-[(R)-2-(2,5-dimethoxy-phenyl)-1-methyl-2-oxo-ethyl]-2,2,2-trifluoro-acetamide and commercially available thiophene-2-carbonyl chloride, followed by hydrolysis of the trifluoroacetamide group and hydrochloride salt formation. The title compound was prepared in 24% overall yield. ¹³C-NMR (400 MHz, DMSO, d₆): δ ppm 17.81 (CH), 25 (CH₂), 30.47 (CH₂), 34.75 (CH₂), 35.46 (CH₂), 46.84 (CH), 55.84 (CH), 55.87 (CH), 66.76 (CH₂), 77.97 (CH), 113.91 (CH), 114.03 (CH), 122.86, 126.36, 150.87, 150.90. MS (APCI) m/z 280 (M+H).

### EXAMPLE 13

### (R)-2-[2,5-Dimethoxy-4-(tetrahydro-thiophen-2-ylmethyl)-phenyl]-1-methyl-ethylamine hydrochloride (Compound 13)

The title compound was prepared by the same procedure reported for Example 1 using N-[(R)-2-(2,5-dimethoxy-phenyl)-1-methyl-2-oxo-ethyl]-2,2,2-trifluoro-acetamide and thiophene-2-carbonyl chloride, followed by hydrolysis of the trifluoroacetamide group and hydrochloride salt formation The title compound was prepared in 30% overall yield. ¹H NMR (400 MHz, DMSO, d₆): δ ppm 1.09 (d, 3H), 1.50-1.75 (m, 4H), 2.70-2.76 (m, 4H), 3.36 (m, 1H), 3.56 (m, 1H), 3.71 (m, 7H), 3.99 (m, 1H), 6.78 (s, 1H), 6.83 (s, 1H), 8.10 (bs, 3H). MS (APCI) m/z 296 (M+H).

### EXAMPLE 14

### 2-(2,5-Dimethoxy-4-methylsulfanylmethyl-phenyl)-1-methyl-ethylamine; hydrochloride

The title compound was prepared by the same procedure used to prepare example 4 using 1-bromo-4-bromomethyl-2,5-dimethoxy-benzene. ¹H NMR (400 MHz, DMSO, d₆): δ ppm 1.12 (d, 3H), 2.06 (s, 2H), 2.72 (m, 1H), 2.92 (m, 1H), 2.50 (m, 1H), 3.63 (s, 2H), 3.74 (m, 6H), 6.85 (s, 1H), 6.91 (s, 1H), 8.08 (bs, 3H). MS (APCI) m/z 256 (M+H). CHN analysis for C₁₃H₂₁NO₂S+1HCl: Calculated: C 53.50, H 7.60, N 4.80; Found: C 53.59, H 7.62, N 4.76

### EXAMPLE 15

### 2-(4-Methanesulfinylmethyl-2,5-dimethoxy-phenyl)-1-methyl-ethylamine trifluoroacetate

To a solution of 2-(2,5-dimethoxy-4-methylsulfanylmethyl-phenyl)-1-methyl-ethylamine hydrochloride (0.80 g, 2.7 mmol) and triethylamine (0.30 g, 3.0 mmol) in 50 mL of DCM was added di-tert-butyl dicarbamate (0.89 g, 4.1 mmol). The reaction mixture was stirred overnight at room temperature and the volatiles were removed under reduced pressure. The residue was purified by flash chromatography (SiO₂, hexanes-ethyl acetate gradient) to give 0.6 g of white solid. To a solution of this compound in 20 mL of DCM at 0° C. was added m-chloroperoxybenzoic acid (0.29 g, 1.69 mmol). The reaction mixture was stirred at 0° C. for 1 h and then allowed to warm to room temperature and stirred for an additional 1 h. The reaction mixture was then partitioned between a saturated aqueous solution of bicarbonate and dichloromethane. The organic layer was separated, dried (anhydrous MgSO₄) and concentrated under reduced pressure to give a yellow residue. This was dissolved in 10 mL of DCM and 5 mL of trifluoroacetic acid. The solution was stirred for 1 h at room temperature and then concentrated to give a solid ¹H-NMR (400 MHz, DMSO, d₆): δ ppm 1.13 (d, 3H), 2.49 (s, 2H), 2.74 (m, 1H), 2.88 (m, 1H), 3.44 (m, 1H), 3.75 (s, 3H), 3.77 (m, 3H), 3.96 (m, 1H), 4.04 (m, 1H), 6.89 (s, 1H), 6.95 (s, 1H), 7.94 (bs, 3H). MS (APCI) m/z 272 (M+H).

### EXAMPLE 16

### 2-[4-(2-Methanesulfinyl-ethyl)-2,5-dimethoxy-phenyl]-1-methyl-ethylamine hydrochloride (Compound 16)

To a solution of 2-[2,5-dimethoxy-4-(2-methylsulfanyl-ethyl)-phenyl]-1-methyl-ethylamine hydrochloride (0.50 g, 1.6 mmol) in DCM (20 mL) was added triethylamine (0.33 g, 3.3 mmol) followed by di-tert-butyl carbonate (0.43 g, 1.97 mmol). The reaction mixture was stirred for 3 h, the volatiles were removed under reduced pressure, and then the residue was purified by flash chromatography (SiO₂, hexanes-ethyl acetate gradient) to give 0.45 g of a solid, which was dissolved in dichloromethane (40 mL) and cooled down to 0° C. To this solution was added 3-chloroperoxybenzoic acid (0.23 g, 1.34 mmol) and the reaction mixture was stirred for 2 h. The reaction mixture was then concentrated, the residue was purified by flash chromatography (SiO₂, dichloromethane-methanol 5%) to give a solid. ¹H NMR (DMSO, d₆): δ ppm 1.05 (d, 3H), 1.37 (s, 9H), 2.68 (s, 3H), 2.69-2.96 (m, 6H), 3.75 (m, 1H), 3.78 (s, 3H), 3.79 (s, 3H), 6.60 (d, NH), 6.81 (s, 1H), 6.88 (s, 1H). LCMS (+APCI) m/z 386 (M+H). The solid was dissolved in dichloromethane (20 mL) and trifluoro acetic acid (20 mL) and stirred for 2 h. The volatiles were removed under reduced pressure, the residue was diluted with DCM (50 mL) and water (50 mL), neutralized by addition of a saturated solution of NaHCO₃, and the organic material was extracted with DCM (3×50 mL). The organic layers were combined, dried (anhydrous MgSO₄), and concentrated to give 14 mg of an oil which was converted to the titled compound by method B. LCMS (+APCI) m/z 286 (M+H).

### EXAMPLE 17

### (R)-2-(4-Benzyl-2,5-dimethoxy-phenyl)-1-methyl-ethylamine hydrochloride (Compound 17)

(R)-2-(4-Benzyl-2,5-dimethoxy-phenyl)-1-methyl-ethylamine; hydrochloride was prepared by a multiple step synthetic procedure.

N-[(R)-2-(4-Benzoyl-2,5-dimethoxy-phenyl)-1-methyl-ethyl]-2,2,2-trifluoro-acetamide

Aluminum chloride (0.55 g, 4.1 mmol) was suspended in DCM (25 mL). N-[(R)-2-(2,5-Dimethoxy-phenyl)-1-methyl-ethyl]-2,2,2-trifluoro-acetamide (0.4 g, 1.4 mmol) was added. The reaction was cooled to 0° C. Next, benzoyl chloride (0.58 g, 4.1 mmol) was added slowly. The ice bath was removed. After stirring overnight at room temperature, ice was added to quench the reaction. The layers were separated. The aqueous layer was extracted with DCM (×2). The organic layers were combined, washed with 1N HCl, water, and saturated NaHCO₃, dried with anhydrous MgSO₄, filtered, and concentrated. Purification was performed using flash chromatography (10% ethyl acetate in hexanes) to give a white solid: 0.33 g (61%). MS (APCI) m/z 396 (M+H)

N-[(R)-2-(4-Benzyl-2,5-dimethoxy-phenyl)-1-methyl-ethyl]-2,2,2-trifluoro-acetamide

N-[(R)-2-(4-Benzoyl-2,5-dimethoxy-phenyl)-1-methyl-ethyl]-2,2,2-trifluoro-acetamide (0.31 g, 0.78 mmol) was dissolved in 7 mL of trifluoroacetic acid. Next, triethylsilane (0.45 g, 3.9 mmol) was added via syringe. After stirring overnight at room temperature, saturated NaHCO₃ solution was added drop wise until the solution remained alkaline. The product was extracted with ethyl acetate, dried with anhydrous MgSO₄, and concentrated. The crude product was triturated with hexanes and collected by vacuum filtration to yield a white solid. Purification was performed using flash chromatography (10% ethyl acetate in hexanes) to give a white solid: 0.22 g (74%). MS (APCI) m/z 382 (M+H)

(R)-2-(4-Benzyl-2,5-dimethoxy-phenyl)-1-methyl-ethylamine hydrochloride (Compound 17)

N-[(R)-2-(4-Benzyl-2,5-dimethoxy-phenyl)-1-methyl-ethyl]-2,2,2-trifluoro-acetamide (0.21 g, 0.55 mmol) was dissolved in methanol (25 mL). Next, a 15% NaOH solution (15 mL) was added. The reaction was stirred at room temperature overnight. Solvent was removed and product was extracted with DCM, dried with anhydrous MgSO₄, and concentrated to yield a white solid which was dissolved in anhydrous ethyl ether. To this solution was added 1.0M solution of hydrogen chloride in ethyl ether. The precipitate that formed was collected by vacuum filtration to give a white solid: 0.11 g. ¹H-NMR (400 MHz, DMSO, d₆): δ ppm 1.12 (m, 3H), 2.67-2.73 (m, 1H), 2.86-2.91 (m, 1H), 3.37-3.40 (m, 1H), 3.70 (s, 3H), 3.74 (s, 3H), 3.88 (s, 2H), 6.83 (s, 1H), 6.87 (s, 1H), 7.14-7.28 (m, 5H), 7.95 (bs, 3H). MS (APCI) m/z 286 (M+H). CHN analysis for C₁₈H₂₃NO₂.1.0HCl.0.2H₂O: Calculated: C 66.43, H 7.56, N 4.30; Found: C 66.40, H 7.43, N 4.29.

### EXAMPLE 18

### (R)-2-[2,5-Dimethoxy-4-(4-methoxy-benzyl)-phenyl]-1-methyl-ethylamine hydrochloride (Compound 18)

By the same general procedure described for the preparation of Example 17, (R)-2-[2,5-Dimethoxy-4-(4-methoxy-benzyl)-phenyl]-1-methyl-ethylamine hydrochloride was prepared using 4-methoxybenzoyl chloride. ¹H-NMR (400 MHz, DMSO, d₆): δ ppm 1.12 (m, 3H), 2.67-2.73 (m, 1H), 2.87-2.92 (m, 1H), 3.37-3.40 (m, 1H), 3.70 (s, 6H), 3.73 (s, 3H), 3.81 (s, 2H), 6.81-6.83 (m, 4H), 7.12-7.15 (m, 2H), 8.05 (bs, 3H). MS (APCI) m/z 316 (M+H). CHN analysis for C₁₉H₂₅NO₃.1.0HCl: Calculated: C 64.86, H 7.45, N 3.98; Found: C 64.48, H 7.50, N 3.95.

### EXAMPLE 19

### (R)-2-[2,5-Dimethoxy-4-(2-methoxy-benzyl)-phenyl]-1-methyl-ethylamine hydrochloride (Compound 19)

By the same general procedure described for the preparation of Example 17, (R)-2-[2,5-dimethoxy-4-(2-methoxy-benzyl)-phenyl]-1-methyl-ethylamine hydrochloride was prepared using 2-methoxybenzoyl chloride. ¹H-NMR (400 MHz, DMSO, d₆): δ ppm 1.12 (m, 3H), 2.69-2.74 (m, 1H), 2.88-2.92 (m, 1H), 3.32-3.41 (m, 1H), 3.65 (s, 3H), 3.73 (s, 3H), 3.82 (m, 5H), 6.73 (s, 1H), 6.81-6.84 (m, 2H), 6.91-6.93 (d, 1H), 6.96-6.98 (d, 1H), 7.16-7.20 (t, 1H), 8.04 (bs, 3H). MS (APCI) m/z 316 (M+H). CHN analysis for C₁₉H₂₅NO₃.1.0HCl: Calculated: C 64.86, H 7.45, N 3.98; Found: C 64.56, H 7.47, N 4.01.

### EXAMPLE 20

### (R)-2-[2,5-Dimethoxy-4-(3-methoxy-benzyl)-phenyl]-1-methyl-ethylamine hydrochloride (Compound 20)

By the same general procedure described for the preparation of Example 17, (R)-2-[2,5-dimethoxy-4-(3-methoxy-benzyl)-phenyl]-1-methyl-ethylamine; hydrochloride was prepared using 3-methoxybenzoyl chloride. ¹H-NMR (400 MHz, DMSO, d₆): δ ppm 1.12 (m, 3H), 2.68-2.73 (m, 1H), 2.88-2.93 (m, 1H), 3.86-3.41 (m, 1H), 3.71 (s, 6H), 3.74 (s, 3H), 3.85 (s, 2H), 6.72-6.74 (m, 1H), 6.79-6.68 (m, 2H), 6.84-6.87 (m, 2H), 7.15-7.19 (m, 1H), 8.04 (bs, 3H). MS (APCI) m/z 316 (M+H). CHN analysis for C₁₉H₂₅NO₃.1.0HCl: Calculated: C 64.86, H 7.45, N 3.98; Found: C 64.72, H 7.47, N 4.01.

### EXAMPLE 21

### (R)-2-[4-(4-Chloro-benzyl)-2,5-dimethoxy-phenyl]-1-methyl-ethylamine hydrochloride (Compound 21)

By the same general procedure described for the preparation of Example 17, (R)-2-[4-(4-Chloro-benzyl)-2,5-dimethoxy-phenyl]-1-methyl-ethylamine hydrochloride was prepared using 4-chlorobenzoyl chloride in step 3. ¹H-NMR (400 MHz, DMSO, d₆): δ ppm 1.12 (m, 3H), 2.68-2.74 (m, 1H), 2.88-2.93 (m, 1H), 3.39-3.41 (m, 1H), 3.72 (s, 6H), 3.87 (s, 2H), 6.85 (s, 1H), 6.89 (s, 1H), 7.22-7.25 (m, 2H), 7.29-7.32 (m, 2H), 8.06 (bs, 3H). MS (APCI) m/z 320 (M+H). CHN analysis for C₁₈H₂₂ClNO₂.1.0HCl: Calculated: C 60.68, H 6.51, N 3.93; Found: C 60.67, H 6.58, N 4.03.

The compounds of formula (I) can be incorporated into various types of ophthalmic formulations for delivery to the eye (e.g., topically, intracamerally, or via an implant). The compounds of formula (I) are preferably incorporated into topical ophthalmic formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound of formula (I) in a physiologically acceptable isotonic aqueous buffer. Further, the ophthalmic solution may include an ophthalmologically acceptable surfactant to assist in dissolving the compound of formula (I). Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the compound of formula (I) in a hydrophilic base prepared from the combination of, for example, carbopol-974, or the like, according to the published formulations for analogous ophthalmic preparations; preservatives and tonicity agents can be incorporated.

The compounds of formula (I) are preferably formulated as topical ophthalmic suspensions or solutions, with a pH of about 4 to 8. The compounds of formula (I) will normally be contained in these formulations in an amount 0.01 to 5% (w/v), but preferably in an amount of 0.1 to 2% (w/v). Thus, for topical presentation 1 to 2 drops of these formulations would be delivered to the surface of the eye 1 to 4 times per day according to the discretion of a skilled clinician.

The compounds of formula (I) can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α₁ antagonists (e.g. nipradolol), α₂ agonists (e.g., iopidine and brimonidine), miotics (e.g., pilocarpine and epinephrine), prostaglandin analogs (e.g., latanoprost, travaprost, unoprostone, and compounds set forth in U.S. Pat. Nos. 5,889,052; 5,296,504; 5,422,368; and 5,151,444, “hypotensive lipids” (e.g., lumigan and compounds set forth in U.S. Pat. No. 5,352,708), and neuroprotectants (e.g., compounds from U.S. Pat. No. 4,690,931, particularly eliprodil and R-eliprodil, as set forth in WO 01/85152, and appropriate compounds from WO94/13275, including memantine.

The following methods can be used to characterize the compounds of the present invention.

### Method 1

### 5-HT₂ Receptor Binding Assay

In order to determine the relative affinities of serotonergic compounds at the 5-HT₂ receptors, their ability to compete for the binding of the agonist radioligand [¹²⁵I]DOI to brain 5-HT₂ receptors is determined as described below with minor modification of the literature procedure [Neuropharmacology, 26, 1803 (1987)]. Aliquots of post mortem rat or human cerebral cortex homogenates (400 μl) dispersed in 50 mM TrisHCl buffer (pH 7.4) are incubated with [¹²⁵I]DOI (80 pM final) in the absence or presence of methiothepin (10 μM final) to define total and non-specific binding, respectively, in a total volume of 0.5 ml. The assay mixture is incubated for 1 hour at 23° C. in polypropylene tubes and the assays terminated by rapid vacuum filtration over Whatman GF/B glass fiber filters previously soaked in 0.3% polyethyleneimine using ice-cold buffer. Test compounds (at different concentrations) are substituted for methiothepin. Filter-bound radioactivity is determined by scintillation spectrometry on a beta counter. The data are analyzed using a non-linear, iterative curve-fitting computer program [Trends Pharmacol. Sci., 16, 413 (1995)] to determine the compound affinity parameter. The concentration of the compound needed to inhibit the [¹²⁵I]DOI binding by 50% of the maximum is termed the IC₅₀ or Kᵢ value.

### Method 2

### 5-HT₂ Functional Assay: Calcium Mobilization

The receptor-mediated mobilization of intracellular calcium ([Ca²⁺]ᵢ) was studied using the Fluorescence Imaging Plate Reader (FLIPR) instrument. Rat vascular smooth muscle cells, A7r5, were grown in a normal media of DMEM/10% FBS and 10 μg/ml gentamycin. Confluent cell monolayers were trypsinized, pelleted, and re-suspended in normal media. Cells were seeded in a 50 μL volume at a density of 20,000 cells per well in a black wall, 96-well tissue culture plate and grown for 2 days. On the day of the experiment, one vial of FLIPR Calcium Assay Kit dye was re-suspended in 50 ml of a FLIPR buffer consisting of Hank's Balanced Salt Solution (HBSS), 20 mM HEPES, and 2.5 mM probenecid, pH 7.4. Cells were loaded with the calcium-sensitive dye by addition of an equal volume (50 μl) to each well of the 96-well plate and incubated with dye for 1 h at 23° C. Typically, test compounds were stored at 25 μM in 50% DMSO/50% Ethanol solvent. Compounds were diluted 1:50 in 20% DMSO/20% Ethanol. For dose-response experiments, compounds were diluted 1:50 in FLIPR buffer and serially diluted 1:10 to give a 5- or 8-point dose-response curve.

At the beginning of an experimental run, a signal test was performed to check the basal fluorescence signal from the dye-loaded cells and the uniformity of the signal across the plate. The basal fluorescence was adjusted between 8000-12000 counts by modifying the exposure time, the camera F-stop, or the laser power. The instrument settings for a typical assay were as follows: laser power 0.3-0.6 W, camera F-stop F/2, and exposure time 0.4 sec. An aliquot (25 μl) of the test compound was added to the existing 100 μl dye-loaded cells at a dispensing speed of 50 μl/sec. Fluorescence data were collected in real-time at 1.0 sec intervals for the first 60 sec and at 6.0 sec intervals for an additional 120 sec. Responses were measured as peak fluorescence intensity minus basal and where appropriate were expressed as a percentage of a maximum 5-HT-induced response.

### EXAMPLE 22

The above procedures were used to generate the data shown in Table 1.

TABLE 1
5-HT2 Receptor Binding and Functional Data

| | IC₅₀ | EC₅₀ | Efficacy |
|---|---|---|---|
| Example | (nM) | (nM) | (Eₘₐₓ %) |
| 1 | 33 | 1100 | 24 |
| 2 | 12 | 1060 | 23 |
| 3 | 0.92 | 78 | 53 |
| 4 | 1.1 | 94 | 51 |
| 5 | 0.18 | 37 | 21 |
| 6 | 0.28 | 22 | 45 |
| 7 | 1.8 | 91 | 32 |
| 8 | 23 | 890 | 30 |
| 9 | 16 | 4900 | 37 |
| 10 | 25 | 600 | 52 |
| 11 | 0.36 | 120 | 55 |
| 12 | 2.4 | 82 | 73 |
| 13 | 4.8 | 560 | 81 |
| 14 | 0.64 | 68 | 52 |
| 15 | 620 | 7600 | 37 |
| 16 | 25 | 870 | 40 |
| 17 | 0.37 | 310 | 43 |
| 18 | 0.19 | 220 | 23 |
| 19 | 0.71 | 660 | 22 |
| 20 | 0.17 | 257 | 53 |
| 21 | 0.16 | 963 | 39 |

### Method 3

### Acute IOP Response in Lasered (Hypertensive) Eyes of Conscious Cynomolgus Monkeys

Intraocular pressure (IOP) can be determined with an Alcon Pneumatonometer after light corneal anesthesia with 0.1% proparacaine. Eyes are washed with saline after each measurement. After a baseline IOP measurement, test compound is instilled in one 30 μL aliquot to the right eyes only of nine cynomolgus monkeys. Vehicle is instilled in the right eyes of six additional animals. Subsequent IOP measurements are taken at 1, 3, and 6 hours.

### EXAMPLE 23

The above method was used to determine the IOP lowering efficacy of Compounds 3 and 4. The results are shown in Table 2.

TABLE 2
IOP Efficacy.

| | | | % IOP change | | |
|---|---|---|---|---|---|
| Example | Dose (μg) | Baseline | 1 hr | 3 hr | 6 hr |
| 3 | 300 | 38.6 | −5.2 | −16.3 | −21.5 |
| 4 | 300 | 35.1 | −3.5 | −14.8 | −16.1 |

The following topical ophthalmic formulations are useful according to the present invention administered 1-4 times per day according to the discretion of a skilled clinician.

### EXAMPLE 24

| Ingredients | Amount (wt %) |
|---|---|
| Compound of formula (I) | 0.1-2 |
| Hydroxypropyl methylcellulose | 0.5 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8-7.4 |
| Purified water | q.s. to 100 |

### EXAMPLE 25

| Ingredients | Amount (wt %) |
|---|---|
| Compound of formula (I) | 0.1-2 |
| Methyl cellulose | 4.0 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8-7.4 |
| Purified water | q.s. to 100 |

### EXAMPLE 26

| Ingredients | Amount (wt %) |
|---|---|
| Compound of formula (I) | 0.1-2 |
| Guar gum | 0.4-6.0 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8-7.4 |
| Purified water | q.s. to 100 |

### EXAMPLE 27

| Ingredients | Amount (wt %) |
|---|---|
| Compound of formula (I) | 0.1-2 |
| White petrolatum and mineral oil and | Ointment |
| lanolin | consistency |
| Dibasic sodium phosphate (anhydrous) | 0.2 |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8-7.4 |

We claim:

1. A method for lowering and controlling normal or elevated IOP, which comprises administering a composition comprising a pharmaceutically acceptable carrier and a pharmaceutically effective amount of a compound of the formula: 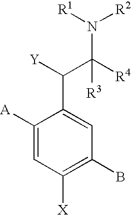
wherein
R¹ and R² independently are H or C₁₋₄alkyl;
R³ and R⁴ independently are H, C₁₋₄ alkyl, or R³, R⁴ and the carbon atom to which they are attached can form a cyclopropyl ring;
Y is H, C₁₋₄alkyl, or OR⁵;
R⁵ is H or C₁₋₄ alkyl;
A and B independently are OH, C₁₋₃alkoxy, OS(O)₂W, OC(═O)W, or OC(═O)NW²W³;
W is C₁₋₄alkyl;
W² and W³ independently are H or C₁₋₄alkyl;
X is CH(OH)R⁶, C(═O)R⁶, (CH₂)ₙZ, or (CH₂)ₘZ′;
R⁶ is C₁₋₄ alkyl, (CH₂)ₘ₂SR⁸, or (CH₂)ₘ₂OR⁸;
n is 1-4;
m is 2-4;
m² is 1-4;
Z is OCF₃, CN, S(O)ₙ₂R⁶, C(O)NR⁷R⁶, C(C═O)R⁶, CH(OH)R⁶, SO₂NR⁷R⁸, NR⁶R⁹, CO2R⁶, S(O)ₙ₂CH₂CH(OH)R⁶, S(O)ₙ₂CH₂C(═)O)R⁶, S(O)ₙ₂CH₂CO2R⁶, S(O)ₙ₂CH₂C(═O)NR⁷R⁸, aryl, heterocyclyl, or heteroaryl;
Z′ is OH or OR⁶;
n is 0 or 1;
R⁷ is H, C₁₋₄alkyl, OH, or OCH₃; and
R⁸ and R⁹ independently are H or C₁₋₄alkyl.

2. The method of claim 1 wherein
R¹, R² are H;
R³ and R⁴ are independently H or C₁alkyl;
Y is H or OR⁵;
R⁵ is H or C₁alkyl;
A and B are independently OH, C₁alkoxy, or OC(═O)W;
W is C₁₋₄alkyl;
X is (CH₂)ₙZ or (CH₂)ₘZ′;
n is 1-4;
m is 2-4;
Z is OR⁶, OCF₃, S(O)ₙ₂R⁶, aryl, heterocyclyl or heteroaryl;
Z′ is OR⁶;
R⁶ is C₁₋₄ alkyl, (CH₂)ₘSR, or (CH₂)ₘOR;
heterocyclyl or heteroaryl is: 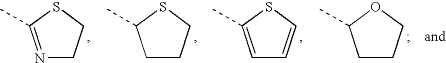
n² is 0.

3. The method of claim 2 wherein the compound is selected from the group consisting of:

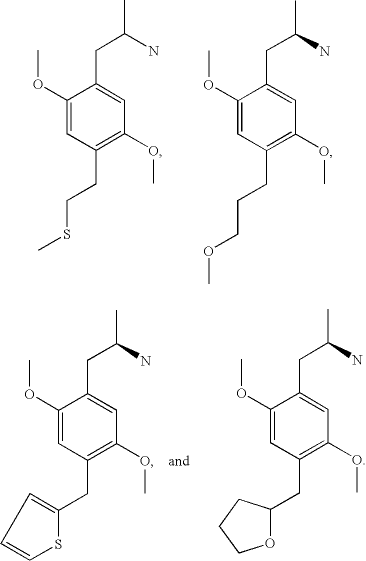

4. The method of claim 1 wherein the composition is a topically administered ophthalmic composition and the pharmaceutically effective amount of the compound is 0.1-2% (w/v).

5. A method for treating glaucoma, which comprises administering a composition comprising a pharmaceutically acceptable carrier and a pharmaceutically effective amount of a compound of the formula: 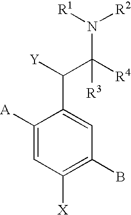
wherein
R¹ and R² independently are H or C₁₋₄alkyl;
R³ and R⁴ independently are H, C₁₋₄ alkyl, or R³, R⁴ and the carbon atom to which they are attached can form a cyclopropyl ring;
Y is H, C₁₋₄alkyl, or OR⁵;
R⁵ is H or C₁₋₄ alkyl;
A and B independently are OH, C₁₋₃alkoxy, OS(O)₂W, OC(═O)W, or OC(═O)NW²W³;
W is C₁₋₄alkyl;
W² and W³ independently are H or C₁₋₄alkyl;
X is CH(OH)R⁶, C(═O)R⁶, (CH₂)ₙZ, or (CH₂)ₘZ′;
R⁶ is C₁₋₄ alkyl, (CH₂)ₘ₂SR⁸, or (CH₂)ₘ₂OR⁸;
n is 1-4;
m is 2-4;
m² is 1-4;
Z is OCF₃, CN, S(O)ₙ₂R⁶, C(O)NR⁷R⁸, C(C═O)R⁶, CH(OH)R⁶, SO₂NR⁷R⁸, NR⁶R⁹, CO2R⁶, S(O)ₙ₂CH₂CH(OH)R⁶, S(O)ₙ₂CH₂C(═)O)R⁶, S(O)ₙ₂CH₂CO2R⁶, S(O)ₙ₂CH₂C(═O)NR⁷R⁸, aryl, heterocyclyl, or heteroaryl;
Z′ is OH or OR⁶;
n² is 0 or 1;
R⁷ is H, C₁₋₄alkyl, OH, or OCH₃; and
R⁸ and R⁹ independently are H or C₁₋₄alkyl.

6. The method of claim 5 wherein
R¹, R² are H;
R³ and R⁴ are independently H or C₁alkyl;
Y is H or OR⁵;
R⁵ is H or C₁alkyl;
A and B are independently OH, C₁alkoxy, or OC(═O)W;
W is C₁₋₄alkyl;
X is (CH₂)ₙZ or (CH₂)ₘZ′;
n is 1-4;
m is 2-4;
Z is OR⁶, OCF₃, S(O)ₙ₂R⁶, aryl, heterocyclyl or heteroaryl;
Z′ is OR⁶;
R⁶ is C₁₋₄ alkyl, (CH₂)ₘSR, or (CH₂)ₘOR;
heterocyclyl or heteroaryl is: 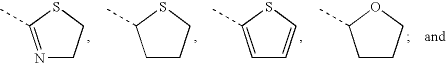
n² is 0.

7. The method of claim 6 wherein the compound is selected from the group consisting of:

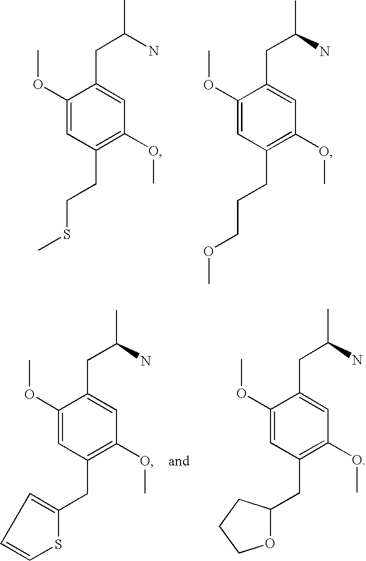

8. The method of claim 5 wherein the composition is a topically administered ophthalmic composition and the pharmaceutically effective amount of the compound is 0.1-2% (w/v).
